(19) United States
(12) Patent Application Publication
(10) Pub. No.: US 2025/0213204 A1
(43) Pub. Date: Jul. 3, 2025

(54) DIGITAL HEALTH ROBOTIC SYSTEM FEATURING DIGITAL HEALTH ROBOT DEVICES AND RELATED METHODS

(71) Applicant: Lyons Global, LLC, Granite Bay, CA (US)

(72) Inventors: Duane Lyons, Granite Bay, CA (US); Bita Lyons, Granite Bay, CA (US)

(21) Appl. No.: 19/008,253

(22) Filed: Jan. 2, 2025

Related U.S. Application Data
(60) Provisional application No. 63/616,836, filed on Jan. 2, 2024.

(51) Int. Cl. A61B 5/00; G05D 1/622; G05D 105/80; G05D 107/60; G16H 80/00; H04L 12/18; H04L 65/403

(52) U.S. Cl. CPC A61B 5/7465; A61B 5/7475; G05D 1/622; G16H 80/00; H04L 12/1831; H04L 65/403; G05D 2105/80; G05D 2107/65

(57) ABSTRACT

A communications platform to facilitate communication between patients and doctors in a remote setting using digital health robot is provided. The digital health robot includes measurement systems to simulate a live examination. A video Visit may be initiated at behest of the patient where doctors may review live results and provide direct feedback to patients.

### FIELD OF THE INVENTION

The present invention relates generally to an integrated healthcare medical examination system to facilitate doctor and patient interactions. More particularly, the present invention provides a medical platform that incorporates digital health robot for doctors to manage live examinations and observation of patients remotely and improve telehealth diagnostics and delivery care models.

### BACKGROUND OF THE INVENTION

Patient-doctor interactions are crucial for ensuring accurate diagnoses and effective treatment plans. To determine the root of the problem, doctors may inquire about the patient's current medical issues and concerns. However, most doctor-patient interactions occur within hospitals or urgent care centers, which may have overcrowding issues. In such conditions, patients may wait for their doctor hours past their scheduled appointment. Even if patients visit their doctor, their concerns may need more medical examinations which may not be addressed during that short visit. Additionally, some patients may be apprehensive about going to hospitals or medical facilities. To remedy these problems, telehealth services was introduced over the last decade. With telehealth solutions, patients may speak to doctors directly from their home or nearest location or their comfort place via internet.

Existing telehealth solutions generally include video conferencing tools and medical examination tools. However, these solutions allow physicians to perform live medical examinations, make diagnoses, and deliver care to patients. These solutions can be utilized by the clinical team or the patient to perform medical tests directly while the doctor guides them through the video visit. Current solutions are also deficient in offering on-demand appointments, where doctors may be accessible to patients on an as-needed basis. Therefore, there exists a need for a platform that allows doctors to facilitate live medical examinations and video visits with on-demand appointment availability.

### SUMMARY OF THE INVENTION

The present invention provides a medical communication and diagnostic systems (“digital health robots”) that may be operable to assist doctors in examinations, monitoring and managing patients remotely. The digital health robots of the present invention include a guidance system, mobile communication functionality, and medical device systems. Physicians may control the digital health robots when performing examinations and conducting patient telehealth visits through a medical communications network that is in telecommunication with the digital health robots, software applications executed on authorized computing devices, and a host computing system that administers the network (the “communications platform”). In some embodiments, digital health robots may be configured to operate using different profiles. It is to be appreciated that throughout the disclosure, the term “doctor” may refer to any of the following: physician, medical practitioner, clinician, health professional, surgeon, consultant, medical doctor, pediatrician, and specialist.

The medical communication and diagnostic systems of the present invention enable healthcare providers to deliver conventional medical care with the benefits of digital assistance. Healthcare facilities and others seeking to improve their healthcare facilities stand to benefit from digital health solutions provided by the system disclosed herein. By removing the geographical barriers that limit classical in-person healthcare, digital health solutions have the potential to improve accessibility, reduce costs, improve clinical outcomes, mitigate staffing difficulties, and improve patient experience metrics without sacrificing the standard of care historically delivered via in-person consultation.

The present invention provides a digital medical that uses digital information and communication technologies to enable access to healthcare services remotely bringing the potential to transform classical healthcare systems into digital health solutions. The platform includes digital health software, digital health robots, and mobile computing platforms. The platform may additionally include AI-based technologies to enhance diagnostics, providing real-time insights and personalized care recommendations. For example, AI features can automate the medical intake form process, triage the patient, and guide the patient to the appropriate doctor. Additionally, AI-based tools may be integrated into the platform that can support enhanced decision-making, enabling more efficient medical triage and consultation workflows.

The present invention integrates advanced artificial intelligence (AI) functionality into the digital health services platform to enhance diagnostic accuracy, streamline workflows, and improve patient care. This invention leverages AI to support key operational and clinical processes, ensuring an efficient, accessible, and comprehensive healthcare delivery system.

The integration of AI into the digital health platform includes a robust training and deployment strategy. This involves aggregating and processing diverse healthcare data to develop machine learning models tailored to medical applications. Historical healthcare data, device readings, and consultation transcripts are anonymized and encrypted to maintain compliance with data privacy laws such as the Health Insurance Portability and Accountability Act (HIPAA). This preparation ensures that the data used in AI model training is secure and ethically managed.

State-of-the-art machine learning frameworks, such as TensorFlow and PyTorch, may be utilized to train AI models capable of performing various diagnostic and administrative tasks. The development process involves collaboration with domain experts to define benchmarks that align with clinical standards, ensuring the AI meets the rigorous demands of medical applications. This collaboration ensures that the AI models are not only technically robust but also clinically relevant.

Once trained, AI modules may be embedded into the digital health platform, enabling smooth interaction with user dashboards, integrated medical devices, and communication protocols. The modular design allows AI systems to function cohesively within the broader healthcare ecosystem, providing real-time support during patient consultations and administrative workflows. To handle computationally intensive tasks, cloud-based AI services may be employed, ensuring the system is scalable and can support growing patient and provider demands.

An iterative feedback mechanism may be used to ensure the continuous improvement of AI functionalities. During deployment, healthcare professionals interact with AI-generated insights, providing valuable feedback that is used to refine the models over time. This validation process ensures that the AI remains accurate and reliable, adapting to new medical data and evolving clinical practices. Regular updates to AI models are conducted, incorporating fresh data to maintain relevance and performance excellence.

The platform may be designed with intuitive interfaces that allow both healthcare providers and patients to interact effortlessly with AI features. For providers, this includes dashboards that display AI-generated insights, such as or patient summaries, in a clear and actionable format. Patients benefit from interfaces that simplify onboarding, symptom reporting, and access to care. These enhancements ensure that users of all technical proficiencies can effectively leverage the system's AI capabilities.

The integration of AI into the platform yields numerous benefits across clinical and operational domains. Workflows for doctors and staff are streamlined through automation, reducing time spent on routine tasks such as documentation and patient triage.

The AI-integration into the system may also reduce administrative burdens by automating documentation processes, such as the generation of SOAP (Subjective, Objective, Assessment, Plan) notes and patient intake forms. This allows healthcare providers to dedicate more time to direct patient care, improving overall service quality and satisfaction.

The AI-integrated digital health platform that enhances the efficiency, accuracy, and accessibility of healthcare services. By combining robust data preparation, advanced machine learning techniques, seamless integration, and iterative validation, the system ensures high-performance AI functionality that aligns with the needs of modern healthcare providers and patients alike.

The medical platform has a plurality of profile users with distinct administrative access and credentials, including Super Admin, Local Admin, CMO, Staff, Doctor, and Patient with role-specific dashboards. The Super Admin role may have the broadest access and include the roles of creating and assigning digital health robots, connecting medical devices, creating healthcare facilities, Local Admin, and CMO. The Super Admin profile activates the other profiles and activates their administrative access, credentials, and intra-system capabilities.

The Super Admin profile may create multiple Local Admin and Multiple CMO roles per healthcare facilities. Local Admins perform operational functionalities such as registering staff and patients. CMOs approve and activate registered doctors' roles. The staff role assists with managing patients, medical intake forms, appointment management, and other patient-related tasks. The patient role provides access to a digital health dashboard, where they can view patient information, medical records, appointments, follow-up tasks, and financial management. All roles have access to secure messaging and notifications. Per clinical requirements, the medical platform may involve a remote digital health robot for medical examinations, as described herein. The medical platform communicates with the robot via a local network (e.g., WiFi router or cellular base station) and through an Internet Service Provider (ISP) to a server hosting the medical platform, utilizing Internet Protocol (IP).

In some embodiments, the medical platform may include a user portal that is operable to provide doctors and patients access to the digital health robots, a software application operable to interface with the digital health robots, and/or patient health records, and each user may have unique access to information when logging in to the communications platform to access and control the digital health robots. The various profile types (super administrators, local administrators, chief medical officers (CMO), staff, doctors, and patients) may provide a user with a unique dashboard corresponding to the control and access provided to the user profile type through the integrated online medical platform. For example, the doctor dashboard may display the patient's medical information through a digital health robot or a software application with the communications platform and execute on a mobile computing device, a desktop computing device, or other computing systems. The staff dashboard may be accessed through a digital health robot or software application. In other embodiments, the dashboard for specific profiles may overlap to facilitate healthcare monitoring, such as overlap between doctor and staff dashboards in medical examinations and monitoring tools.

In some embodiments, a user profile on the communications platform may include stringent security measures to prevent altering and viewing sensitive medical information. For example, if a doctor completes a visit summary and sends it to the patient, other profiles may not have access to alter the message. In some embodiments, these security measures may include two-factor authentication to prevent other users from accessing a person's medical information and healthcare plan. In other embodiments, other forms of authentication may be used to access a user profile and dashboard.

A healthcare facility may utilize the medical platform by integrating its patient records and various data specific to the healthcare facility. A Super Admin can create a profile for the healthcare facility in the medical platform and enable the healthcare facility to use the medical platform by adding their local Admins, CMO, Staff, Doctors, and patients as needed. The Super Admin may then register one or more robot computers and add all the computer's specifications inside the digital health robot to a robot profile. The Super Admin may also add an inventory of all the medical devices to the robot profile. The robot profile may include all the technical specifications of each medical device. The technical specifications may include the MAC address of each medical device, which may each be in electronic communication with a local network either individually or via electronic communication with the digital health robot, enabling communication with the server for the medical platform. Each medical device may be able to share data (e.g., diagnostic data) with the medical platform through MAC address-enabled digital communication.

### Patient Intake and Triage

To enhance the efficiency and accuracy of patient intake and triage, the described digital health platform integrates an AI-powered module designed to streamline this process. The AI module interacts with patients via the digital health robot, collecting symptoms through voice or text input. Using advanced natural language processing (NLP), the system translates these symptoms into structured data, which it may cross-reference with the patient's historical medical data stored on the platform. This NLP functionality may allow the digital health robot to engage patients in conversational exchanges, collecting symptoms, guiding them through the medical intake process, and addressing initial queries. For instance, the conversational agent can ask a patient about their symptoms, follow up with clarifying questions, and summarize the collected information for the doctor's review. This ensures a thorough and patient-friendly intake experience, particularly for individuals who may struggle with traditional forms or technical systems.

Training the NLP models may further involve leveraging datasets derived from telehealth conversations, symptom-intake questionnaires, and publicly available linguistic datasets focused on healthcare contexts. These datasets may be annotated to include medical terminology, conversational patterns, and contextual nuances, ensuring that the models can understand and respond to patient inputs effectively. Techniques such as fine-tuning pretrained language models on domain-specific data are used to improve the agent's ability to recognize medical terms and intent. By leveraging these datasets, the AI learns to recognize the language used by the patient, patterns in patient-reported symptoms and associate them with likely conditions. Continuous learning mechanisms are employed to refine the system's accuracy. For instance, after a doctor evaluates the AI's recommendations during a consultation, the doctor's decisions are fed back into the AI system, allowing it to adjust and improve over time.

This functionality is integrated into the platform's workflow by embedding the AI module into the digital health robot's software. Patients initiate the intake process via the robot's interface, and the collected data is securely transmitted to the doctor's dashboard in real-time. The secure transmission of patient intake data from the digital health robot to the doctor's dashboard in real-time may be achieved through advanced encryption and communication protocols. All patient data is encrypted both in transit and at rest using industry-standard protocols such as Advanced Encryption Standard (AES-256), ensuring that sensitive information remains secure and inaccessible to unauthorized parties during transmission. Communication between the digital health robot and the doctor's dashboard may be further safeguarded by Secure Sockets Layer/Transport Layer Security (SSL/TLS) encryption, providing end-to-end data integrity and confidentiality. To comply with healthcare data privacy laws such as HIPAA, the system employs specialized communication channels designed to handle sensitive medical information.

To enable real-time updates, the platform may utilize low-latency communication protocols such as WebSockets or HTTP/2. These protocols ensure that data is transmitted immediately upon submission, allowing doctors to access and review the information without delay. Once received, the doctor's dashboard organizes the intake data into structured categories, including symptoms, historical medical records, medication usage, and allergies. This categorization provides a logical and easily navigable presentation of patient information. Additionally, the Ai module embedded in the platform enhances the data presentation by offering summary insights and suggested triage recommendations. All symptoms and potential diagnoses are flagged for the doctor's attention, facilitating informed decision-making.

Integration into the platform is achieved by embedding the NLP engine within the robot's software interface and/or patient dashboard. Patients interact with the conversational agent via the robot's touchscreen, voice interface, or both. The collected data is securely transmitted to the doctor's dashboard, where it is presented as a summarized, structured report. The dashboard may also include interactive visualizations, such as charts and graphs, to represent patient metrics like vital signs, making the data easier to interpret. A real-time notification system may alert doctors to new intake submissions or high-priority cases requiring immediate attention. By combining secure data transmission protocols with an intuitive and informative dashboard, the platform ensures that doctors receive comprehensive and actionable patient information efficiently, enabling them to provide timely and effective care.

The digital health robot may include medical examination devices, and transfers the data to the provider dashboard to observe the data and deliver care remotely. The communications platform may be operable to facilitate connections between patients and doctors. In some embodiments, the doctor may employ the digital health robot connected to the communications platform to conduct remote consultations and perform guided examinations. Patients may use the digital health robot to communicate remotely to facilitate the health care visit through the communications platform. For example, the doctor may conduct remote consultations through the communications via the medical platform from their hospital office using the digital health robot present in the patient's home as an interface. In other embodiments, the location of the patient and doctor may be different from those described above. For example, the digital health robot may be present in a hospital clinic where the patient is receiving care, and the doctor will be remote.

In some embodiments, the patient and doctor may each use the digital health platform and use their respective digital health robots to conduct encrypted video consultations. Prior to performing the consultation, the digital health robots may request authentication between the patient and the doctor. In some embodiments, the authentication software may use two-factor authentication.

In such embodiments, there are two sides to digital health users: one can be digital health robots, and the other can be remote clinicians who are performing video consultations between patients and doctors. In these scenarios, the doctor may control the digital health robot remotely. In most embodiments, the digital health robot is positioned at the patient's side, while the doctor is remote. The doctor and the robot may be located in different places. The doctor can add other doctors to the robot's system for multi-specialty consultations, allowing all doctors to view the patient and participate in the live medical examination simultaneously. This integration ensures that patients receive comprehensive, real-time care from multiple specialists, regardless of their location, and facilitates efficient collaboration among healthcare professionals through the digital health platform.

Examinations may be conducted by the doctor through the digital health robot. During video consultations, the doctor may be able to guide the digital health robot to perform a medical examination of the patient. For some medical examinations, the doctor may ask the nurses to help perform them. The digital health robot may perform live streaming of the medical data to doctors from patients. The medical platform and digital health robot may perform pre-diagnostic tasks through the medical devices integrated into the digital health robot. The doctor may perform medical examinations through digital health robot. In some embodiments, a home healthcare nurse or staff nurse may work with patients to use the medical device systems and communicate with the doctor. AI will follow up and write in the visit summary note by summarizing key points of the consultation, automatically generating follow-up recommendations, and noting any additional actions required for the patient, such as scheduling further appointments, tests, or medication reminders.

The medical devices may be operable to provide doctors on the medical platform with a set of medical diagnostics that describe the patient's well-being. In some embodiments, a plurality of medical device systems may be integrated within the digital health robot. Such medical device systems may include but are not limited to, pan-tilt-zoom (PTZ) exam cameras, a digital stethoscope, a digital pulse oximeter, digital blood pressure, digital dermoscopy, digital thermometer, digital weight scale, digital stethoscope, digital glucometer, digital spirometry, digital otoscope, ultrasound, and a digital ECG system. The medical devices in the present invention may be integrated within the digital health robots. Each medical device within the digital health robot may perform as a distinct system. The medical device systems may act as robotic guidance measurement tools that interface with the digital health robot. For example, if a doctor requests to measure a patient's temperature, the thermometer system within the medical platform may initiate and track the patient's temperature in real-time using the thermometer device. The doctor is thereby able to track the patient's temperature throughout the examination process. Each measurement is fully integrated into the doctor dashboard through the digital health platform.

### Real-Time Diagnostic Assistance

The described robot healthcare platform integrates an AI module for real-time diagnostic assistance to enhance the accuracy and efficiency of medical examinations. This module processes data collected from medical devices integrated into the digital health robot. During a patient consultation, the AI module analyzes data in real-time, detecting patterns and anomalies in oxygen saturation levels from the pulse oximeter, or fever conditions from temperature readings. When an anomaly is detected, the system flags it for the doctor's immediate attention, ensuring critical conditions are not overlooked.

This functionality may be integrated into the platform by embedding the AI within the communication interface between the robot and the doctor's dashboard. The analyzed data and AI-generated insights are displayed on the dashboard in a clear, actionable format, such as highlighted anomalies and corresponding recommendations. This seamless integration ensures that doctors can quickly access and act upon diagnostic data, improving clinical outcomes and enhancing the overall efficiency of remote healthcare delivery.

In some embodiments, the doctor may use the communications platform within the doctor dashboard to access the patient's medical information. The patient's medical information may include past medical history, medications, allergies, family history, surgeries, hospitalizations, social history, stored results from medical devices, and past visit summary notes. In other embodiments, the patient's medical information may include other relevant information. AI can collect all of the patient's past medical history and automatically generate SOAP (Subjective, Objective, Assessment, Plan) notes at a level appropriate for the doctor's review. These AI-generated SOAP notes will summarize key clinical data, enabling the doctor to quickly assess the patient's condition and plan appropriate next steps in the care process. This helps ensure that the doctor has a comprehensive and up-to-date view of the patient's health, enhancing clinical decision-making and improving the efficiency of patient management.

In some embodiments, the medical platform may include a Picture Archiving and Communication System (PACS) that allows for secure storage and display of medical images. The system may be used to manage, retrieve, distribute, and present images in the patient's medical records and captured by the digital health robot.

In some embodiments, the staff or patient may transfer confidential documents from the digital health robot through the communications platform to the physician. Confidential documents may range from medical forms to imaging results retrieved from external sources. In other embodiments, the confidential documents may be different from mentioned above.

Doctors may write clinical notes and order plans through a web-based doctor digital health platform interface on a computing device. The platform may provide a dashboard for the doctor to connect with patients, review patient records, direct testing through the patient's digital health robot, conduct video visits with patients, and make medical orders. If a doctor assigns a task, the staff and patient dashboards may display the required tasks and steps to follow. For example, if the doctor refers the patient to another doctor through the doctor dashboard, the patient may see a task on their patient dashboard provided through the digital health platform to complete the task. Staff will also be notified, and AI can assist by generating reminders, offering instructions, and ensuring that all required steps are followed for the task completion. In some embodiments, the patient may read a message on their dashboard through the patient dashboard or digital health robot and communicate with the doctor as needed. AI can also write SOAP notes for doctors when they use the AI features in the robot and platform, summarizing key clinical findings and actions taken. These AI-generated SOAP notes can be stored securely, helping streamline documentation and improving the accuracy of medical records, allowing the doctor to focus on patient care.

The digital health platform may incorporate an AI module capable of automatically generating structured SOAP notes, streamlining documentation during patient-doctor interactions. This functionality significantly reduces the administrative burden on healthcare providers, allowing them to focus more on patient care. The AI may listen to or process transcripts of live consultations between the doctor and patient, identifying and extracting key clinical insights. It may organize this information into the four components of a SOAP note: subjective patient-reported symptoms, objective measurable data from diagnostic devices, a clinical assessment based on findings, and a proposed treatment plan.

To train the AI for this functionality, a large dataset of annotated consultation transcripts may be utilized. These transcripts include detailed examples of properly structured SOAP notes, enabling the AI to learn how to extract relevant information and organize it effectively. The training process relies on natural language processing (NLP) models to interpret medical terminology and contextual characteristics in doctor-patient conversations. Continuous learning mechanisms are employed to improve accuracy over time by incorporating feedback from healthcare providers who review and validate the AI-generated SOAP notes.

The integration of this functionality into the robot healthcare platform may be achieved by embedding the AI module within the doctor's dashboard interface. During consultations, the AI operates in the background, analyzing conversations and device data in real-time during the visit. Once the consultation concludes, the AI presents a draft SOAP note on the doctor's dashboard for review. This note may be editable, allowing the doctor to make corrections or additions before finalizing the documentation. This seamless integration ensures that accurate and comprehensive notes are generated efficiently, enhancing the overall quality of patient records.

### Decision Support

The robot healthcare platform may further integrate an AI-driven decision support system to assist medical personnel in making informed diagnostic and treatment decisions. For example, it may recommend potential diagnoses for a patient presenting with fever and fatigue or suggest appropriate medication dosages based on historical treatment outcomes. This may also include identifying abnormalities in diagnostic images captured by medical devices integrated into the robot such as ultrasound probes or dermoscopy cameras. This functionality enables the platform to process and analyze medical images in real-time, providing doctors with valuable insights during remote consultations. For example, the AI may be able to detect and highlight irregularities such as suspicious skin lesions or abnormal structures in ultrasound scans, flagging them for further review by the clinician. This capability enhances diagnostic accuracy and helps doctors prioritize their focus on areas of concern.

The decision support AI may operate as part of the doctor's dashboard, analyzing patient data in real-time during consultations. The AI-generated recommendations are presented to the doctor through an interactive interface, highlighting potential diagnoses and ranked treatment options. When a medical image is captured, it is automatically processed by the AI module, which analyzes the image and overlays insights or annotations directly onto the doctor's dashboard. The interface may also allow doctors to override or modify suggestions, ensuring the final decision rests with the clinician. This collaboration between AI and doctors may enhance diagnostic accuracy, reduces cognitive workload, and expedites clinical workflows.

In some embodiments, a staff nurse or medical assistant may be assigned to the patient and may log in to their profile on the staff users to the digital health robot. The staff nurse may be operable to communicate with the doctor through the staff dashboard through the digital health platform and on the digital health robot. Staff nurses or medical assistants may be provided with a dashboard that displays the patient's current medical status, ongoing medications, treatment plans, care plans, and other relevant information. In some embodiments, AI can collect Medical Intake information with talking to patients via robot, as described herein.

In some embodiments, administrators may be able to access the digital health robot. Administrator profiles may be limited to specialized tasks facilitating patient and doctor interaction. For example, administrators may assist patients in scheduling appointments, taking payments, and handling user management. In other embodiments, administrators may be able to assist with other tasks.

Patients using the patient dashboard digital health platform may be able to attend on-demand or scheduled appointments. A scheduled appointment can be booked for upcoming dates. In some embodiments, the digital health platform may offer patients the ability to schedule appointments according to available doctors. In other embodiments, the digital health platform may allow patients to start an on-demand appointment. On-demand appointments may be scheduled if the patient needs to have access to the doctor on the same day at the same time the patient or the doctor requested the on-demand visit. In other embodiments, the patient may be able to schedule an on-demand appointment for other reasons.

### Appointment Management and Resource Allocation

In some embodiments, the robot healthcare platform integrates an AI-driven appointment management and resource allocation system to optimize scheduling and resource utilization. This functionality enhances the efficiency of healthcare delivery by predicting appointment times that align with patient needs, doctor availability, and operational constraints such as clinic hours and medical equipment usage. The AI module may evaluate multiple factors, such as patient urgency, geographic proximity, and historical data on appointment durations and patient no-show rates, to recommend optimal scheduling slots. For example, the system may prioritize early appointments for high-risk patients or allocate extended slots for consultations that typically take longer, such as multi-specialty visits.

The AI functionality is integrated into the platform by embedding it within the administrative dashboard used by staff and healthcare providers. The system interfaces with patient and doctor profiles to dynamically suggest appointment slots, which can be viewed and confirmed through the dashboard. For doctors, the dashboard highlights upcoming appointments and associated resource requirements, such as medical devices or support staff. For patients, the system provides flexible scheduling options that match their availability and urgency, enhancing accessibility and satisfaction. This integration results in efficient use of resources, reduces scheduling conflicts, and improves the overall workflow of the system.

In some embodiments, a plurality of management systems is operably integrated into the communications platform. The communications platform may manage the clinic's practices, health care facilities, physicians, staff, patients, administration, and the robot autopilot. In other embodiments, there may be more management systems for any single clinic. For example, the physician management system may review all physicians assigned to the communications platform and direct them toward patients, staff, and administrative services.

In some embodiments, the digital health robots may include integrated medical device systems for performing live examinations of patients through doctor guidance. The doctor may control the digital health robots from their respective location. For example, the doctor, staff, or administrator may utilize the digital health platform at their respective location. In other embodiments, the digital health robot may be operated by doctors, staff, or administrators through a software application executed by a mobile computing device, desktop computer, or other remote computing devices.

It is an aspect of the present invention to provide a communication platform for patients and doctors to communicate in a virtual clinic setting. The communications platform may include encrypted video conferencing between patients and doctors. Doctors can connect to the robot to start the video appointment call with a patient to discuss relevant health information. During the video consultation, the patient may be able to exchange medical records and relevant medical information, while the doctor can review the patient's medical information and confirm any diagnoses through the communications platform. Additionally, the doctor can add other doctors to the robot for multi-specialty consultations, enabling collaborative decision-making during the live medical examination.

The doctor can also add the patient's family members to the robot session as needed, allowing for comprehensive discussions about the patient's care and health history. All participants, including the doctor, other doctors, and family members, can be remote and connect to the robot video visit, facilitating a flexible, collaborative healthcare environment. This setup ensures that the patient receives timely, thorough, and multi-disciplinary care, regardless of the location of the involved healthcare providers or family members.

Another aspect of the present invention is providing a communications platform for doctors to perform live medical examinations on patients in a remote setting. Doctors may guide digital health robots that include diagnostic systems to perform live examination on the patient through the communications platform. Once a visit is completed, patients and doctors may review relevant information within their respective dashboards.

In another aspect, the present invention provides a communications platform for hospital staff, patients, doctors, and administrators to communicate with each other remotely. The platform may include an array of dashboards each tied to a user profile. Each user profile may be assigned a set of permissions to limit access to sensitive medical information. For example, doctors may be assigned permission to view patient's medical information, while administrators may be restricted to patient scheduling, employee records, billing, or user management.

Further aspects and embodiments will be apparent to those having skill in the art from the description and disclosure provided herein.

It is an object of the present invention to facilitate remote healthcare visits between patients and doctors where doctors may perform guided examinations and consultations on an on-demand basis.

It is an object of the present invention to facilitate remote healthcare visits between patients and doctors utilizing digital health platform and robot to perform guided examinations and consultations.

The above-described objects, advantages, and features of the invention, together with the organization and manner of operation thereof, will become apparent from the following detailed description when taken in conjunction with the accompanying drawings, wherein like elements have like numerals throughout the several drawings described herein. Further benefits and other advantages of the present invention will become readily apparent from the detailed description of the preferred embodiments.

### BRIEF DESCRIPTION OF THE DRAWINGS

FIG. 1 provides an exemplary view of a digital health robot according to an embodiment of the present invention.

FIG. 2 provides an exemplary view of a digital health robot, according to an embodiment of the present invention.

FIG. 3 provides a relational diagram of a digital health robot, according to an embodiment of the present invention.

FIG. 4 provides a second relational diagram of a medical communications platform, according to an embodiment of the present invention.

FIG. 5 provides an exemplary view of utilization of digital health robots connecting through a medical communications platform, according to an embodiment of the present invention.

FIG. 6 provides a relational diagram of user profiles, according to an embodiment of the present invention.

FIG. 7 provides a flow diagram of the digital health robot, according to an embodiment of the present invention.

FIG. 7A provides a flow diagram of the digital health robot, according to an embodiment of the present invention.

FIG. 8 provides a second relational diagram of user profiles, according to an embodiment of the present invention.

FIG. 9 provides a second flow diagram of the digital health robot, according to an embodiment of the present invention.

FIG. 10 provides a third flow diagram of the digital health robot, according to an embodiment of the present invention.

FIG. 11 provides a fourth flow diagram of the digital health robot, according to an embodiment of the present invention.

FIG. 12A provides a relational diagram of the patient dashboard, according to an embodiment of the present invention.

FIG. 12B provides a relational diagram of the doctor dashboard, according to an embodiment of the present invention.

### DETAILED DESCRIPTION

Reference will now be made in detail to certain embodiments of the invention, examples of which are illustrated in the accompanying drawings. While the invention will be described in reference to these embodiments, it will be understood that they are not intended to limit the invention. To the contrary, the invention is intended to cover alternatives, modifications, and equivalents that are included within the spirit and scope of the invention. In the following disclosure, specific details are given to provide a thorough understanding of the invention. However, it will be apparent to one skilled in the art that the present invention may be practiced without all of the specific details provided.

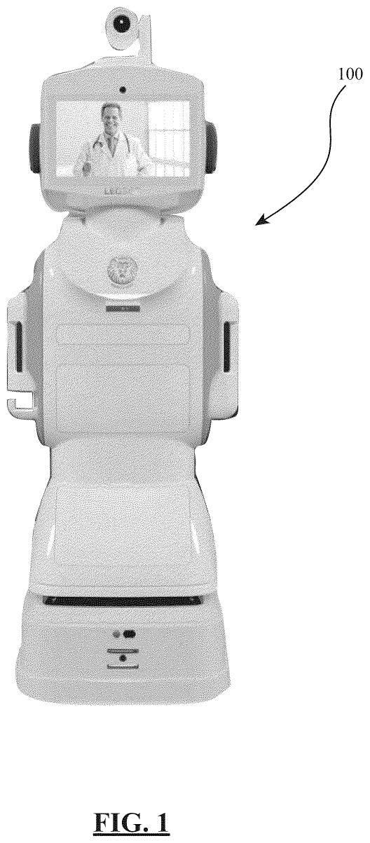

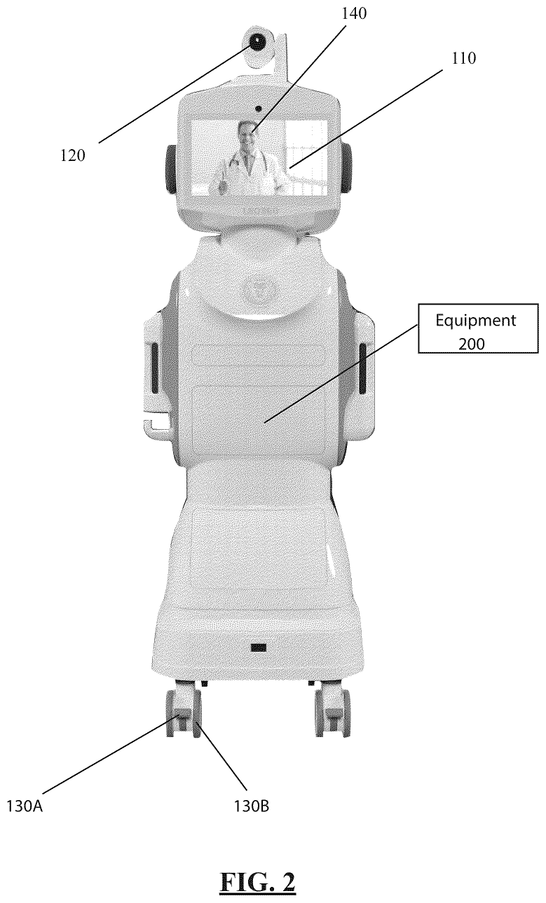

Referring to the drawings wherein like reference characters designate like or corresponding parts throughout the several views and referring particularly to a digital health robot 100 of FIGS. 1-2, it is seen that in this illustrated embodiment, the screen 110 and camera 120 may be used to facilitate communication between doctor 140 and patients 150 in a video call 300. The digital health robot 100 may include wheels 130 for transportation and equipment 200 for live medical examinations. AI-based modules may be integrated into the robot 100 that are operable to dynamically to interact with the patient through the screen 110 during consultations to collect patient information such as symptoms and reporting from the patient. This interview information may be transmitted and displayed to a dashboard for the doctor and/or other medical personnel.

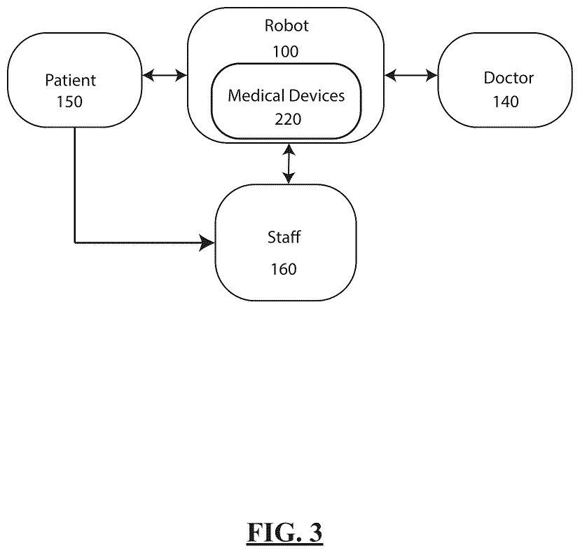

In some embodiments, the digital health robot 100 may communicate with doctors 140, patients 150, and staff 160, as shown in FIG. 3. Staff 160 and doctors 140 may access the peripheral devices 220 to perform measurements on patient 150. Once measurements are performed, the digital health robot 100 may be in communication with a doctor dashboard 440, patient dashboard 450, and staff dashboard 460. AI-driven diagnostics may analyze data from the peripheral devices 220 in real-time, flagging anomalies for immediate review by healthcare professionals through the doctor dashboard 440 and/or patient dashboard 450.

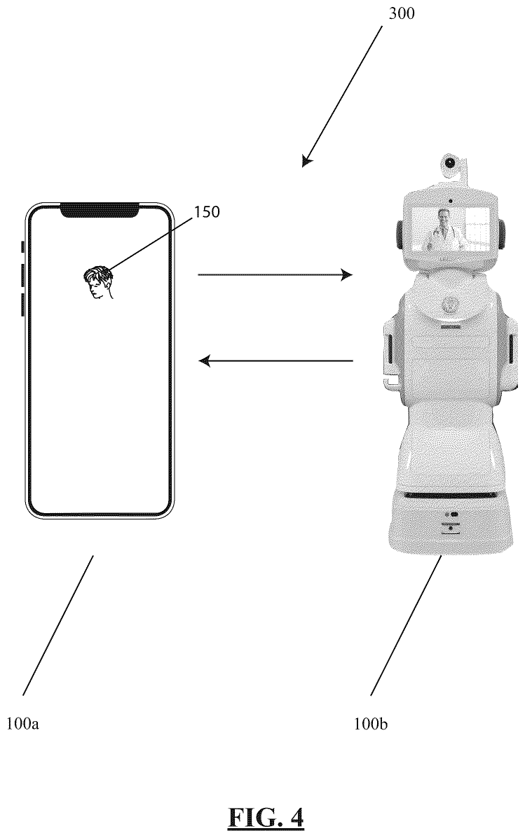

The digital health robot 100 may be operable to start a video call 300 between a patient 150 and a doctor 140 using screen 110, as shown in FIG. 4. In some embodiments, there may be a digital health robot 100 and digital health platform 100a and 100b to facilitate a video call 300. The doctor 140 or other healthcare provider may interface with the digital health robot 100 through a software application executed by a mobile computing device, desktop computing device, or device through the communications platform. The medical platform is in electronic communication with the robot 100, e.g., via connection of the digital health robot to a local network (e.g., via a WiFi router or a cellular base station) and through an Internet Service Provider (ISP) to a server hosting the medical platform 100a using the Internet Protocol (IP). AI conversational agents may assist during the call, interpreting patient responses and generating a summary of key points discussed, which can be directly shared with the doctor in real-time.

### Profiles and Dashboards

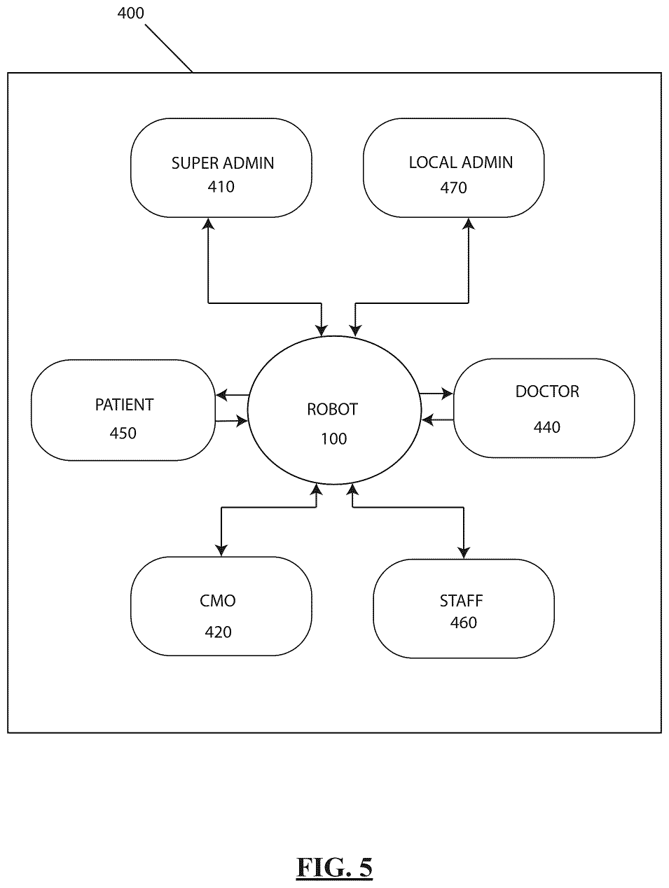

In some embodiments, the communications platform may employ a plurality of user profiles. The platform may include a plurality of user profiles 400. In some examples, the platform may include six distinct types of user profiles each with their own dashboards, as seen in FIG. 5. Each type of profile may use restrictive protocols to limit access to sensitive information. For example, doctor profile 440 and patient profile 450 may share information with each other using digital health platform 100a. Staff profile 460 may also communicate between doctor profile 440 and patient profile 450 using the digital health robot 100. The super admin profiles 410 or local admin profiles 470 may not be able to access patient's 150 medical information or medical tests. In other embodiments, the patient profile 450, doctor profile 440, and staff profile 460 may communicate with each other using the communications platform 100a. Communication between profiles 400 enables a seamless workflow for patients 150, doctors 140, staff 160, and administrators 170. The communication protocols may be sufficient to prevent a breach of security and patient confidentiality. AI features may monitor interactions between profiles for operational efficiency, automatically recommending task optimizations based on historical workflow data.

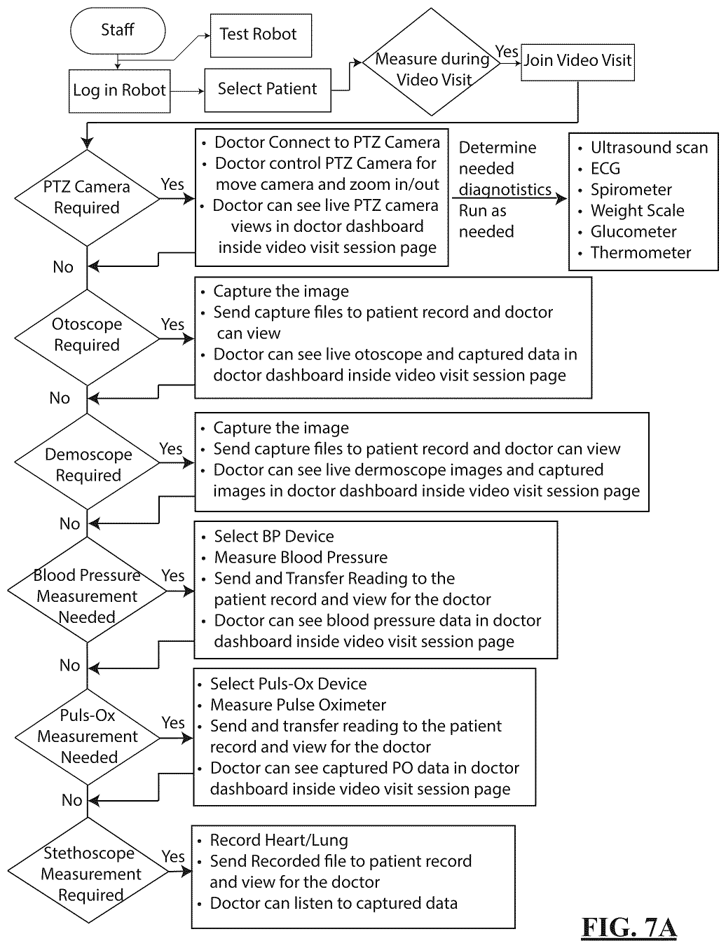

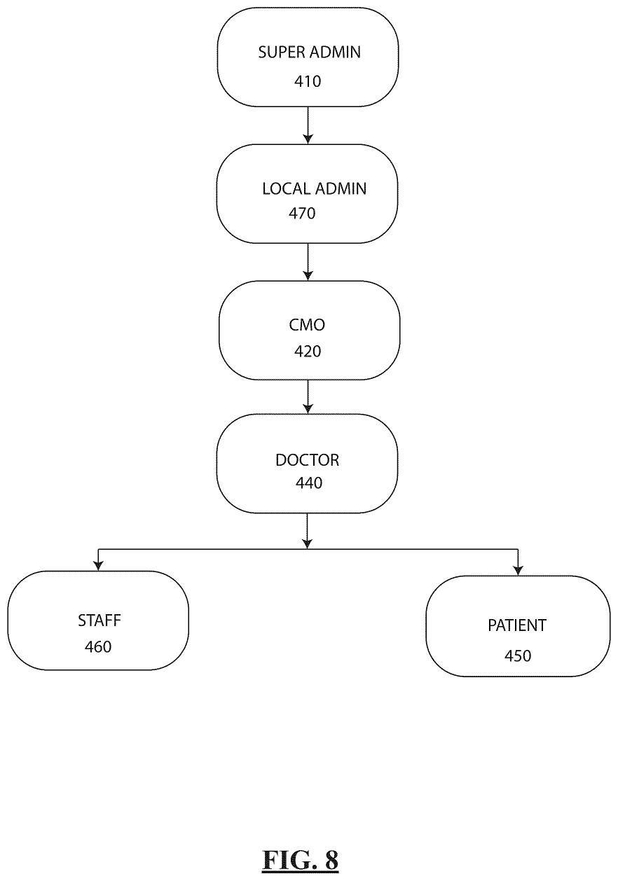

In some embodiments, there may be at least six users of the digital health robot 100 including a super admin profile 410, local admin 470, Chief Medical Officer (CMO) 420, Doctor 440, staff 460, and patient profile 450, as shown in FIG. 8. Super admins 410 may be granted the highest access level within the platforms 100a and 100b, allowing them to grant approvals and register new doctor profiles 440 and CMOs 420. An AI module may provide analytics to super admins, offering insights into platform utilization, security risks, and system performance, ensuring optimal management.

Local admins 470 may be operable to register staff 460 members, manage patients 450, communicate with staff 460 members, and other items. Super admins 410 may be operable to register local admins 470, staff 460, doctors 440, manage patients 450, and assign digital health robots 100 to doctors 440. CMOs 420 may be operable to manage doctors 140 registered on the platform, approve doctors 140, and confirm doctor 140 schedules. Doctors 140 may communicate with staff 160 and patients 150 using digital health robots 100. AI module can assist in automating these processes, such as by pre-validating new patient data entries for accuracy and completeness ensuring that all necessary information fields are filled by either the patient or by survey carried out by medical staff or interactive AI agent.

In some embodiments, each profile registered to the digital health robot 100 or the communication platform may include a dashboard. The digital health robot 100 may employ a dashboard platform 400, where the health robot 100 may communicate with at least six dashboards including a super admin dashboard 410, local admin dashboard 470, patient dashboard 450, doctor dashboard 440, CMO dashboard 420, and a staff dashboard 460. An AI-enhanced dashboard may present role-specific predictive insights, such as patient trends for doctors or operational bottlenecks for administrators.

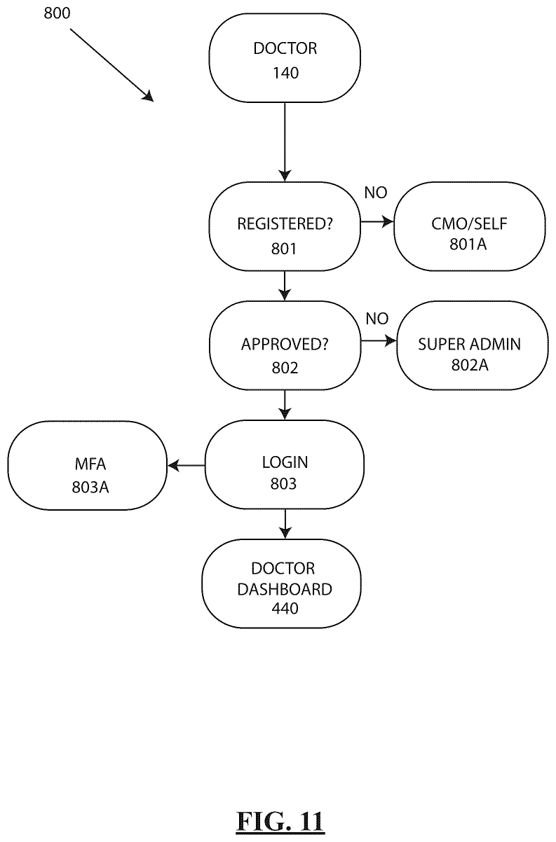

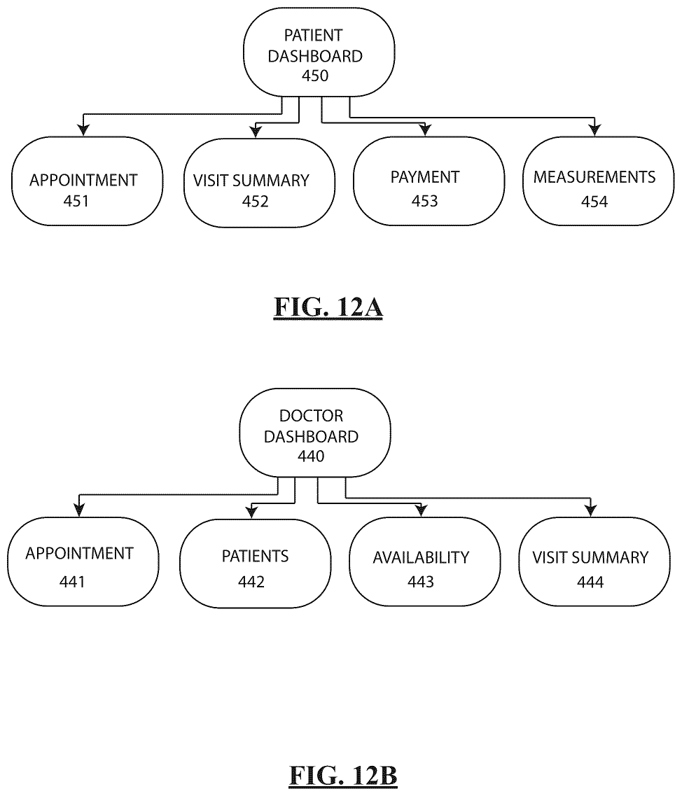

The doctor dashboard 440 may include appointment scheduling 441, patient management 442, availability schedule 443, and visit summaries 444, as shown in FIG. 12A. Patient dashboards 450 may include appointment scheduling 451, visit summary 452, payment methods 453, and measurement data 454, as shown in FIG. 12B. An AI agent may populate the visit summaries with insights extracted from real-time consultations and provide suggested next steps based on similar cases.

Registering users to the platforms 100a and 100b may require approval from super admin profiles 410 and local admins 410. For example, when adding CMOs 420 to the communication platform, process 600 is initiated, as shown in FIG. 9. Firstly, the CMO's account is created by a super admin in step 601. The CMO may then login to the platform in step 602 using multi-factor authentication (MFA) 602A. Once the CMO logs in, approvals 603 and 604 are required by a super admin 410 and doctor 140. Shortly thereafter, the CMO may engage in communication 605 with the doctor 140 either by accessing a doctor dashboard 440 or the like.

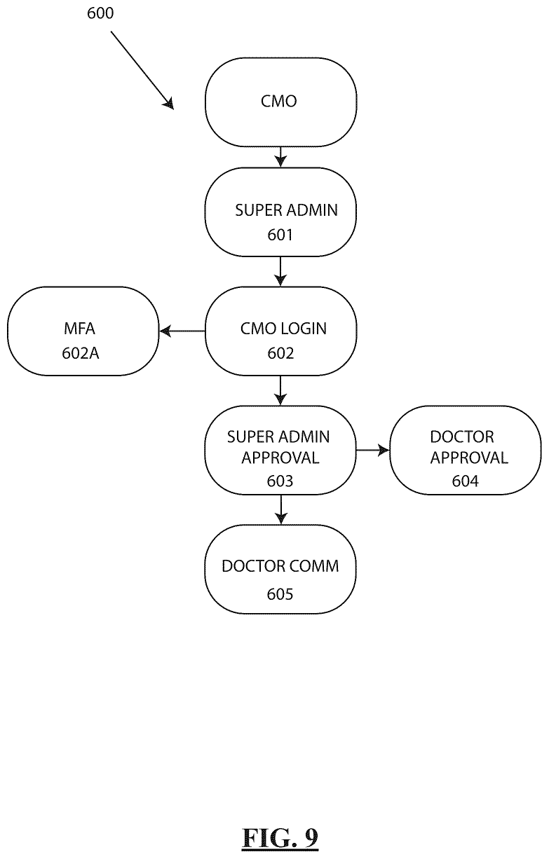

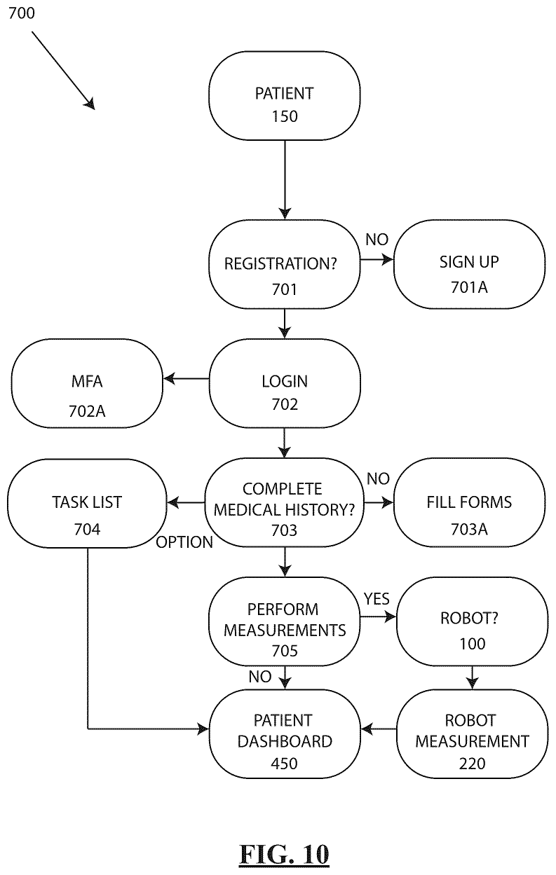

Similar methods are employed when adding patients to the platform 100a and 100b in process 700, as shown in FIG. 10. Patients 150 may be registered 701 by self-signup 701A or through the assistance of staff 160. Patients may then login 702 using MFA 702A and then are asked to complete medical history 703 or move on to a task list 704. If medical history 703 is not completed, patients may complete forms 703A. Once the medical history 703 is completed, patients may perform measurements 705 through a digital health robot 100 which may perform measurements 220 and send over results to a patient dashboard 450. In other embodiments, patients may directly enter the patient dashboard 450 if not needing to perform measurements 705.

In some embodiments, doctors 140 may be added to the health platform 100 through process 800, as shown in FIG. 11. The health platform may verify if the doctor is registered 801, which may prompt the doctor 140 to self-register 801A or request assistance from a CMO 420. The doctor 140 may request approval 802 from a super admin 410 in step 802A, and may proceed to login 803 using MFA 803A. Once logged in 803, the doctor 140 may proceed to the doctor dashboard 440 to complete their required tasks. Required tasks may include reviewing patient measurements, checking appointment times, and the like.

An AI security module may monitor access to the communications platform and review access compliance by analyzing login behaviors and flagging suspicious activities. For example, attempts to authorize features of the communication platform without authorization may be flagged and sent to super admin profiles for review.

### Robot Access

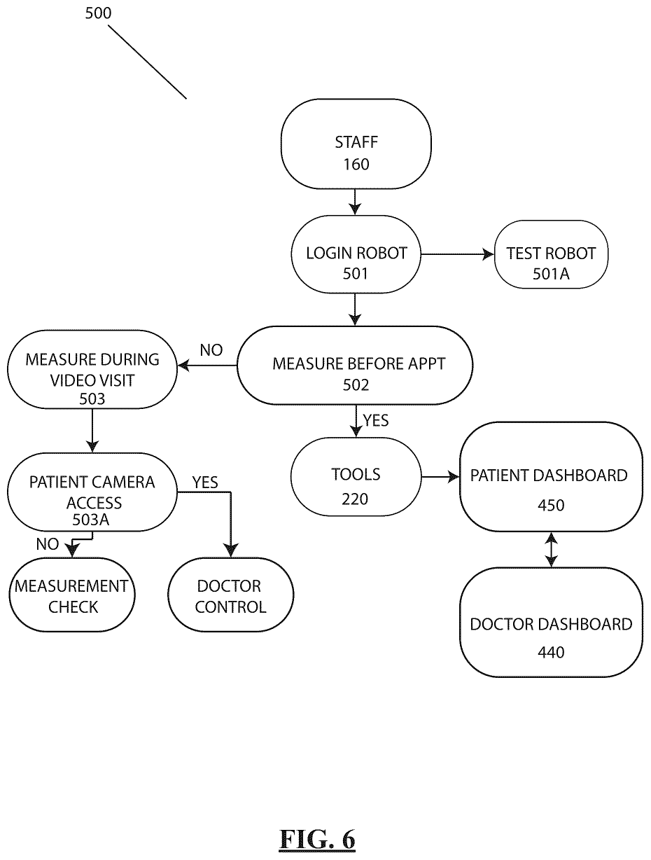

Digital health robots 100 may be accessed by staff 160 through process 500, as shown in FIG. 6. Staff 160 may login 501 to the robot 100, and in some instances may test the robot's functionality 501A. The robot 100 may prompt the staff member 160 to either measure before appointment 502. If the staff member selects yes, the peripheral systems 220 may be activated to perform measurements on the patient 150. Once measurements are completed, the peripheral systems 220 may send the data over to the patient dashboard 450 and the doctor dashboard 440. In other embodiments, the staff member 160 may select measure during video visit 503 where doctors 140 may request for patient camera access 503A. Once camera access 503A is granted, doctors 140 may take control of the digital health robot 100 and perform measurements on the patient 150. In other embodiments, the staff member may deny camera access 503A, and proceed to check measurements. An AI intake agent may guide patients through form completion, automatically extracting relevant information from uploaded documents and prior records to streamline the process.

A patient 150 may log in to their profile 450 on digital health platform 100a. In some embodiments, the patient 150 may use the patient dashboard 450 to schedule an appointment with a doctor 140. Appointments may be categorized as on-demand or scheduled. If a patient 150 schedules an on-demand appointment using digital health, a doctor 140 may be requested for a video visit call 300. Patient may search for doctors 140 by specialty and patient requirement. Once the doctor 140 has been selected by the patient, a video visit 300 may begin using camera 120 on both the digital health platform 100a and the digital health robot 100b (or through the software application) to facilitate a patient 150 and doctor 140 interaction. The AI intake agent may optimize appointment slots, prioritizing high-risk cases and minimizing wait times based on dynamic resource allocation models.

In some embodiments, the digital health platform 100 may be used by patients 150 to communicate with doctors 140 in a telehealth setting. Patients 150 may schedule an appointment with a doctor 140 using screen 110 after logging in to patient profile 450 on the digital health platform 100b. During an appointment, a doctor 140 may use camera 120 to conduct a video visit 300 with patient 150. The doctor 140 may also conduct live examinations of the patient using equipment 200 and medical device systems 220. The patient 150 may transfer confidential documents and information through the digital health platform 100b to the digital health platform 100a. Once a video visit 300 is completed, the doctor 140 may use their user profile and dashboard 440 to conduct a review of the patient's 150 overall health and log any tasks for the patient 150 to complete. The patient 150 may then use their user profile and dashboard 450 to track all upcoming appointments and required tasks to complete such as referrals, follow up, orders and prescription. In other embodiments, the staff 160 may use their staff profile and dashboard 460 to communicate with the doctor 140 for the patient 150 using a digital health robot platform 100.

In some embodiments, doctors 140 may monitor patients 150 using a digital health platform 100a. The digital health platform 100a may include equipment 200 to use for remote examinations. During a video visit call 300, the patient may use the digital health robot 100b in a remote location. The doctor 140 may then remotely guide equipment 200 to perform live medical examinations using digital health platform 100a. In other embodiments, the doctor or other healthcare provider may remotely guide equipment 200 to perform live medical examinations through a software application executed by a mobile computing device, desktop computing device, or other remote computing device through the communications platform. An AI module may be embedded within equipment 200 to analyze measurements in real-time, offering preliminary diagnosis suggestions and flagging deviations from normal patient diagnostic results and measurements.

The doctor 140 may control the camera 120 on the digital health robot 100b from the digital health platform 100a (or through the software application) to visualize the patient 150 during a video Visit 300. By controlling the camera 120 on the digital health robot 100, the doctor 140 may visualize the live examination of the patient 150 using equipment 200 and determine the appropriate course of action. For example, during a stethoscope (not shown) test, the doctor 140 may control the camera 120 towards the patient 150. This will allow the doctor 140 to verify if the stethoscope (not shown) is placed in the appropriate location for the examination.

### Equipment Overview

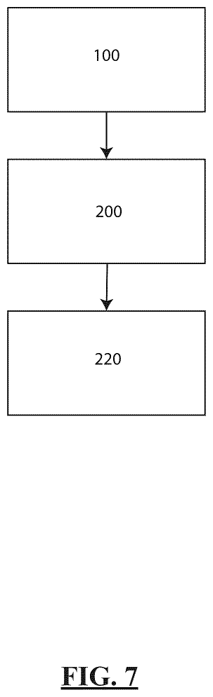

Equipment 200 may include a wide range of instruments used to measure the readings of a patient 150. In some embodiments, equipment 200 may include PTZ exam cameras, a stethoscope, a digital pulse oximeter, digital blood pressure monitor, digital dermoscopy, digital thermometer, digital weight scale, digital stethoscope, digital glucometer, digital spirometry, digital otoscope, ultrasound, and a digital 12 Lead EKG (peripheral systems). In other embodiments, equipment 200 may include other instruments used to measure biometric indicators of patients. The doctor may select specific diagnostic operations to perform during the examination with the peripheral systems 220. As shown in FIG. 7A, the doctor may select one or more exam protocols as shown in the flow chart. An AI module integrated with these tools enhance their functionality, such as providing automated calibration checks to optimize data quality before examination.

Equipment 200 in digital health robot 100 may include a set of peripheral systems 220, as shown in FIG. 7. Peripheral systems 220 may be digitally operated and controlled to provide real time feedback to the doctor 140 during a video visit 300. During a live examination, feedback to the doctor 140 may be relayed to the screen 110 on the digital health platform 100a during a video visit 300 from the digital health robot 100b (or through the software application).

In some embodiments, the peripheral systems 220 on medical platform 100 may be operable to perform live examinations of the patient 150 through the guidance of the doctor 140. Live examinations may include vital sign measurement, blood pressure, pulse oximetry, ECG, and other medical exams. Once examinations are completed, the peripheral systems 220 may be operable to relay information to doctor dashboard 440 and patient dashboard 450. The AI module can recommend specific tests based on the patient's reported symptoms, dynamically updating recommendations as new data is collected during the session.

### Document Transfer

During video visit consultation 300, the patient 150 may send documents to the doctor 140 using the digital health robot 100b (or through the software application). Examples of some documents may include medical forms, imaging scans, past medical history, and other health information. Doctors 140 may be able to access the documents through the digital health robot 100a and perform a medical exam accordingly. In other embodiments, the patient 150 may send documents to the doctor 140 outside of a video visit call 300 using digital health robot 100b. For example, if the doctor requests lab results or immunization history, the patient 150 may send over the documents through the digital health platform 100. The AI intake agent can capture images of the documents, perform optical character recognition (OCR), process the documents, and extract and categorize information into relevant sections of the patient's profile.

Transferring of medical documents may use two forms of encryption from the patient 150 and doctor 140. Medical documents may include consent forms, treatment agreements, and privacy disclosures to comply with legal and ethical standards. Therefore, the protection of these documents is critical when establishing a relationship between doctor 140 and patient 150. The AI intake agent may include an anomaly detection system operable to monitor document transfer activities, ensuring compliance with HIPAA standards and preventing unauthorized access.

When a doctor 140 sends unsigned documents using the digital health platform 100a, the patient 150 may transfer signed documents and authorize the transfer using two-factor authentication. In other embodiments, the authorization of transfer may be completed using the combination of two-factor authentication. Once a doctor 140 receives signed documents from a patient 150, said signed documents may be saved to a secure database in the communications platform for later access.

### Communication Protocols

Following a video visit call 300, a patient 150 may communicate with the doctor 150 using a messaging system (not shown) on the communications platform and accessible through the digital health robot 100. The messaging system may use standardized protocols to ensure confidentiality and integrity of patient information. Some examples of messaging protocols may include HTTPs, SSL/TLS. In other embodiments, the messaging protocols may be different from those mentioned above. The AI intake agent may assist in these interactions by generating concise, context-aware responses to patient queries and summarizing ongoing discussions for the doctor's reference.

In some embodiments, the digital health platform 100a may be operable to provide referrals to other specialized doctors 140 when requested by patients 150 after an initial video visit 300. For example, if a patient 150 attends a video Visit with a doctor 140 for a physical appointment, the doctor 140 may use the digital health platform 100a to refer the patient 150 to another doctor 140 to meet their needs. The AI intake agent may be operable evaluate the patient's medical history and symptoms, suggesting specialists with expertise in the required area to medical personnel to facilitate the referral.

In some embodiments, the communications platform may use a database accessible through the digital health platform 100a to store medical records of patients 150 for later access by doctors 140. In other embodiments, administrators 170 may be able to access the database to retrieve patient's 150 billing details.

In some embodiments, the digital health robot 100 is operable to be controlled by a doctor 140. In other embodiments, USB devices may be in communication with the communication platform and may be operable to receive data from a plurality of peripheral devices and transmit the data directly from robot to the doctor dashboard and patient data dashboard. These peripheral devices may include blood pressure monitors, glucometers, pulse oximeters, thermometers, weight scales, ultrasounds, 12 Lead EKG, exam cameras, otoscopes, dermoscopic, spirometers, and stethoscope. In other embodiments, the USB device may receive other peripheral devices.

The digital health robot 100 may include a navigation system 130A that may be operated by the doctor 140 during a video visit call 300. In some embodiments, navigation system 130A may be operable to move the digital health robot 100 in any direction laterally with respect to the patient. For example, the digital health robot 100 may be controlled by the doctor 140 and travel laterally and execute rotational movements based on the position of the patient 150 or the staff member 160. The navigation system 130A may be operable to avoid obstacles by using sensors (not shown) integrated within the medical platform 100. Some sensors may include gyroscopes, accelerometers, LIDAR, radar, computer vision, infrared, and other sensors. In other embodiments, the digital health robot may not have a navigation system 130A, and may be manually operated using wheels 130B by the staff 160 or the patient 150. In some embodiments, the health robot 100 may include an AI module that is operable to analyze other computer vision data to identify obstacles and may interact with a pathfinding algorithm optimize the robot's movements, avoiding obstacles and ensuring precise positioning for medical examinations.

It is to be understood that variations, modifications, and permutations of embodiments of the present invention, and uses thereof, may be made without departing from the scope of the invention. It is also to be understood that the present invention is not limited by the specific embodiments, descriptions, or illustrations or combinations of either components or steps disclosed herein. The embodiments were chosen and described in order to best explain the principles of the invention and its practical application, to thereby enable others skilled in the art to best utilize the invention and various embodiments with various modifications as are suited to the particular use contemplated. Although reference has been made to the accompanying figures, it is to be appreciated that these figures are exemplary and are not meant to limit the scope of the invention. It is intended that the scope of the invention be defined by the claims appended hereto and their equivalents.

1. A digital health communication and diagnostic system, comprising:
a. at least one digital health robot including
i. a mobile communication device configured to enable encrypted video conferencing between a healthcare provider and a patient,
ii. a plurality of integrated medical device systems operable to perform live medical examinations, and
iii. a navigation system configured for remote control by the healthcare provider;
b. a medical communications platform facilitating remote operation of said at least one digital health robot by said healthcare provider operable to
i. provide a healthcare provider dashboard displaying patient medical information and real-time data from the medical device systems, and
ii. facilitate secure data exchange between the healthcare provider and the patient; and
c. A host computing system administrating said network and interconnected with software applications executed on authorized computing devices, wherein said at least one digital health robot is configurable to operate using distinct profiles, each profile having role-specific dashboards and administrative access levels;

2. (canceled)

3. The system of claim 1, wherein each digital health robot includes medical examination devices operable to transfer diagnostic data to a digital dashboard for remote healthcare delivery by doctors.

4. (canceled)

5. (canceled)

6. The system of claim 1, wherein the communications platform supports multi-specialty consultations by connecting multiple healthcare providers through separate digital communications devices to the same digital health robot session.

7. (canceled)

8. The system of claim 1, further comprising a patient dashboard configured to display visit summaries and follow-up tasks through said at least one digital health robot.

9. The system of claim 1, wherein the digital health robot includes voice-activated controls for patient interaction.

10. (canceled)

11. (canceled)

12. (canceled)

13. (canceled)

14. (canceled)

15. (canceled)

16. (canceled)

17. (canceled)

18. (canceled)

19. The platform of claim 1, wherein the digital health robot includes an AI-powered module to process patient intake information through natural language processing.

20. (canceled)

21. (canceled)

22. (canceled)

23. The platform of claim 1, wherein the AI module generates structured Subjective, Objective, Assessment, and Plan (SOAP) notes summarizing the consultation for the doctor's review.

24. A digital health robot, comprising:
a. a housing configured for mobility, including a navigation system operable to move the robot and avoid obstacles using integrated sensors;
b. a plurality of medical device systems integrated within the housing, each configured to collect patient diagnostic data during live medical examinations; and
c. a communication system configured to enable encrypted video conferencing between a healthcare provider and a patient and to transmit diagnostic data in real time to a remote healthcare provider dashboard.

25. (canceled)

26. The robot of claim 24, wherein the robot is equipped with obstacle detection sensors.

27. The robot of claim 24, wherein the integrated medical devices include one or more of pan-tilt-zoom (PTZ) exam cameras, digital stethoscopes, pulse oximeters, blood pressure monitors, digital dermoscopes, thermometers, weight scales, glucometers, spirometers, otoscopes, ultrasound devices, and digital ECG systems.

28. (canceled)

29. (canceled)

30. (canceled)

31. (canceled)

32. (canceled)

33. The robot of claim 24, wherein the AI module provides predictive maintenance alerts for the robot's medical devices and operational components.

34. (canceled)

35. A method for performing remote healthcare using a remote healthcare platform comprising a digital health robot and a communications platform, the method comprising:
a. initiating an encrypted video conferencing session between a healthcare provider and a patient using the digital health robot;
b. remotely controlling the digital health robot to perform live medical examinations on the patient using integrated medical device systems; and
c. transmitting real-time diagnostic data from the digital health robot to a healthcare provider dashboard for review and assessment by the healthcare provider.

36. (canceled)

37. The method of claim 35, wherein the medical examination includes utilizing one more integrated medical devices including pan-tilt-zoom (PTZ) exam cameras, digital stethoscopes, pulse oximeters, blood pressure monitors, digital dermoscopes, thermometers, weight scales, glucometers, spirometers, otoscopes, ultrasound devices, and digital ECG systems.

38. The method of claim 35, wherein the transmitted diagnostic data is analyzed by an AI module to identify potential anomalies.

39. (canceled)

40. The method of claim 35, wherein the digital health robot is remotely controlled by the doctor during the examination.

41. (canceled)

42. (canceled)

43. The method of claim 35, wherein the AI module provides interactive guidance to the patient during the examination using natural language processing.

44. The method of claim 35, further comprising generating a structured consultation summary, including Subjective, Objective, Assessment, and Plan (SOAP) notes, using an AI module based on the diagnostic data and doctor-patient interaction.

45. The method of claim 35, wherein the AI module dynamically suggests additional diagnostic tests based on real-time analysis of collected patient data.

46. (canceled)

47. (canceled)

48. The method of claim 35, wherein the AI module generates visualizations of diagnostic data, including graphs and charts, for the doctor's dashboard.

49. (canceled)

50. The method of claim 35, wherein the AI module automatically categorizes patient data into structured fields for the doctor's review and electronic medical record integration.

51. (canceled)

52. (canceled)

53. (canceled)

54. (canceled)

55. (canceled)

56. (canceled)

57. (canceled)
